(19) United States
(12) Patent Application Publication
(10) Pub. No.: US 2024/0254576 A1
(43) Pub. Date: Aug. 1, 2024

(54) A method for manufacturing direct reduced iron

(71) Applicant: ArcelorMittal, Luxembourg (LU)

(72) Inventors: George TSVIK, Valparaiso, IN (US); Dmitri BOULANOV, East Chicago, IN (US); Jon REYES RODRIGUEZ, AVILES Asturias (ES); Odile CARRIER, Metz (FR); Sarah SALAME, Maizieres les Metz (FR); José BARROS LORENZO, Maizieres les Metz (FR); Marcelo ANDRADE, East Chicago, IN (US); Dennis LU, East Chicago, IN (US)

(21) Appl. No.: 18/559,909

(22) PCT Filed: May 18, 2021
(86) PCT No.: PCT/IB2021/054259

§ 371 Date: Nov. 9, 2023

(51) Int. Cl. C21B 13/00; C25B 1/04; C25B 15/08

(52) U.S. Cl. CPC C21B 13/0086; C21B 13/0066; C21B 13/0073; C25B 15/081; C21B 2100/26; C21B 2100/282; C25B 1/04

(57) ABSTRACT

A method for manufacturing direct reduced iron wherein oxidized iron is reduced in a direct reduction furnace by a reducing gas, the oxidized iron being first mixed with biochar to form a solid compound and the solid compound is charged into the direct reduction furnace.

The invention is related to a method for manufacturing Direct Reduced Iron (DRI) and to a DRI manufacturing equipment.

### BACKGROUND

Steel can be currently produced through two main manufacturing routes. Nowadays, the most commonly used production route consists in producing pig iron in a blast furnace, by use of a reducing agent, mainly coke, to reduce iron oxides. In this method, approx. 450 to 600 kg of coke is consumed per metric ton of pig iron; this method, both in the production of coke from coal in a coking plant and in the production of the pig iron, releases significant quantities of CO2.

The second main route involves so-called “direct reduction methods”. Among them are methods according to the brands MIDREX, FINMET, ENERGIRON/HYL, COREX, FINEX etc., in which sponge iron is produced in the form of HDRI (Hot Direct Reduced Iron), CDRI (cold direct reduced iron), or HBI (hot briquetted iron) from the direct reduction of iron oxide carriers. Sponge iron in the form of HDRI, CDRI, and HBI usually undergo further processing in electric arc furnaces.

There are three zones in each direct reduction shaft with cold DRI discharge: Reduction zone at top, transition zone at the middle, cooling zone at the cone shape bottom. In hot discharge DRI, this bottom part is used mainly for product homogenization before discharge.

Reduction of the iron oxides occurs in the upper section of the furnace, at temperatures up to 950° C. Iron oxide ores and pellets containing around 30% by weight of Oxygen are charged to the top of a direct reduction shaft and are allowed to descend, by gravity, through a reducing gas. This reducing gas is entering the furnace from the bottom of reduction zone and flows counter-current from the charged oxidized iron. Oxygen contained in ores and pellets is removed in stepwise reduction of iron oxides in counter-current reaction between gases and oxide. Oxidant content of gas is increasing while gas is moving to the top of the furnace.

The reducing gas generally comprises hydrogen and carbon monoxide (syngas) and is obtained by the catalytic reforming of natural gas. For example, in the so-called MIDREX method, first methane is transformed into a reformer according to the following reaction to produce the syngas or reduction gas:

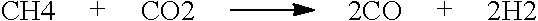

and the iron oxide reacts with the reduction gas, for example according to the following reactions:

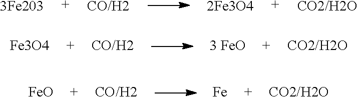

At the end of the reduction zone the ore is metallized.

A transition section is found below the reduction section; this section is of sufficient length to separate the reduction section from the cooling section, allowing an independent control of both sections. In this section carburization of the metallized product happens. Carburization is the process of increasing the carbon content of the metallized product inside the reduction furnace through following reactions:

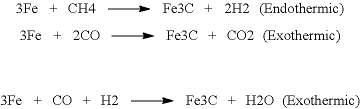

Injection of natural gas in the transition zone is using sensible heat of the metallized product in the transition zone to promote hydrocarbon cracking and carbon deposition. Due to relatively low concentration of oxidants, transition zone natural gas is more likely to crack to H2 and Carbon than reforming to H2 and CO. Natural gas cracking provides carbon for DRI carburization and, at the same time adds reductant (H2) to the gas that increases the gas reducing potential.

### SUMMARY OF THE INVENTION

In view of the considerable increase in the concentration of CO2 in the atmosphere since the beginning of the last century and the subsequent greenhouse effect, it is essential to reduce emissions of CO2 where it is produced in a large quantity, and therefore in particular during DRI manufacturing.

One solution which is currently developed is the progressive increase of the hydrogen content into the reducing gas, in view of reaching a pure hydrogen reducing gas. Following reduction reaction will then occur:

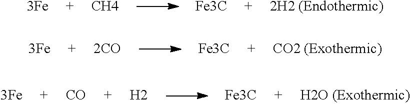

thus releasing harmless H2O instead of the greenhouse gas CO2.

This however implies that the content of carbon into the reducing gas will be reduced and at some point, no more carbon will be injected into the shaft. As explained above this has an impact on the DRI product which will have a smaller and smaller carbon content.

Content of carbon in the DRI product is a key parameter at it plays an important role into the subsequent steps, such as slag foaming at the electric Arc furnace, but it also helps to improve the transportability of the DRI product.

Solutions are already known to increase the carbon content of the product, they mainly consist in injecting hydrocarbons into the shaft, usually CH4, or coke oven gas. But those gases will contribute to increase the carbon footprint of the DRI process which is not in line with the switch to pure H2 reduction.

There is a need for a method allowing to increase carbon content in the DRI product while reducing the carbon footprint of the process.

The present invention provides a method, wherein oxidized iron is reduced in a direct reduction furnace by a reducing gas, said oxidized iron being first mixed with biochar to form a solid compound and said solid compound is charged into said direct reduction furnace.

The method of the invention may also comprise the following optional characteristics considered separately or according to all possible technical combinations:
the biochar is produced by the pyrolysis of biomass,
the solid compound is briquette and/or pellet,
the reducing gas comprises more than 50% in volume of hydrogen,
the reducing gas comprises more than 99% in volume of hydrogen,
the hydrogen of the reducing gas is at least partly produced by electrolysis,
electrolysis is powered by renewable energy,
a top reduction gas is captured at the exit of the direct reduction furnace and subjected to at least one separation step to be split between a CO2-rich gas and an H2-rich gas, said H2-richgas being at least partly used as reduction gas,
the CO2-rich gas is subjected to a methanation step.

### BRIEF DESCRIPTION OF THE DRAWINGS

Other characteristics and advantages of the invention will emerge clearly from the description of it that is given below by way of an indication and which is in no way restrictive, with reference to the appended FIGURES in which:

FIG. 1 illustrates a layout of a direct reduction plant allowing to perform a method according to the invention

Elements in the figures are illustration and may not have been drawn to scale.

### DETAILED DESCRIPTION

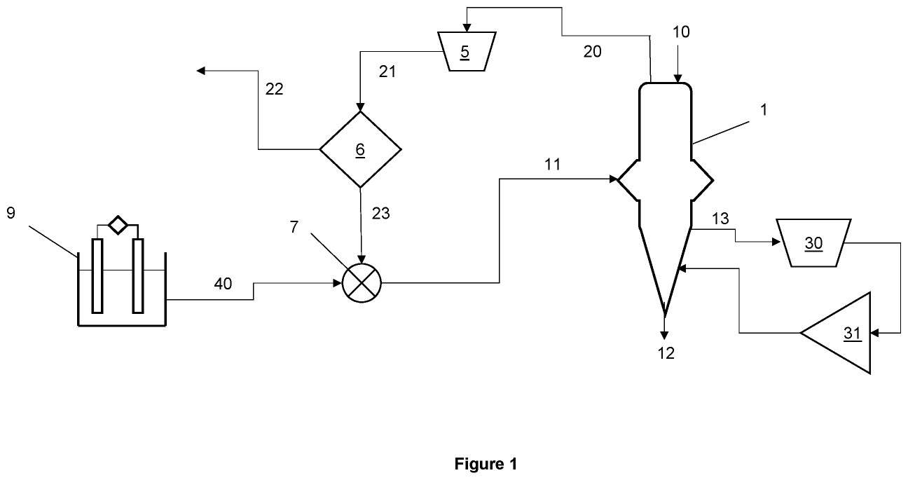

FIG. 1 illustrates a layout of a direct reduction plant allowing to perform a method according to the invention. In said method, the direct reduction furnace (or shaft) 1 is charged at its top with a compound 10 made of a mixture of oxidized iron and biochar. Said compound may have any suitable shape allowing the loading into the furnace, it is preferentially charged in form of briquettes and/or pellets. In a preferred embodiment, the compound 10 comprises from 0.01 to 10% by weight of biochar. By Biochar it is meant a charcoal that is produced by pyrolysis of biomass in the absence of oxygen.

Biomass is renewable organic material that comes from plants and animals. Biomass sources for energy include wood and wood processing wastes-firewood, wood pellets, and wood chips, lumber and furniture mill sawdust and waste, and black liquor from pulp and paper mills, agricultural crops and waste materials-corn, soybeans, sugar cane, switchgrass, woody plants, and algae, and crop and food processing residues, biogenic materials in municipal solid waste-paper, cotton, and wool products, and food, yard, and wood wastes and animal manure and human sewage.

The compound 10 will provide both the iron oxides to be reduced and the necessary carbon-source to carburize the metallized product. In a preferred embodiment, carbon content of the Direct Reduced Iron is set from 0.5 to 3 wt. %, preferably from 1 to 2 wt. % which allows getting a Direct Reduced Iron that can be easily handled and that keeps a good combustion potential for its future use.

Said compound 10 is reduced into the furnace 1 by a reducing gas 11 injected into the furnace and flowing counter-current from the compound 10. Reduced iron 12 exits the bottom of the furnace 1 for further processing, such as briquetting, before being used in subsequent steelmaking steps. Reducing gas, after having reduced iron, exits at the top of the furnace as a top reduction gas 20 (TRG).

A cooling gas 13 can be captured out of the cooling zone of the furnace, subjected to a cleaning step into a cleaning device 30, such as a scrubber, compressed in a compressor 31 and then sent back to the cooling zone of the shaft 1.

In a preferred embodiment, the reducing gas 11 comprises at least 50% v of hydrogen, and more preferentially more than 99% v of H2. An H2 stream 40 may be supplied to produce said reducing gas 11 by a dedicated H2 production plant 9, such as an electrolysis plant. It may be a water or steam electrolysis plant. It is preferably operated using CO₂ neutral electricity which includes notably electricity from renewable source which is defined as energy that is collected from renewable resources, which are naturally replenished on a human timescale, including sources like sunlight, wind, rain, tides, waves, and geothermal heat. In some embodiments, the use of electricity coming from nuclear sources can be used as it is not emitting CO2 to be produced.

In another embodiment, H2 stream 40 may be mixed with part of the top reduction gas 20 to form the reducing gas 11. When operated with natural gas the top reduction gas 20 usually comprises from 15 to 25% v of CO, from 12 to 20% v of CO2, from 35 to 55% v of H2, from 15 to 25% v of H2O, from 1 to 4% of N2. It has a temperature from 250 to 500° C. When pure hydrogen is used as reducing gas, the composition of said top reduction gas will be rather composed of 40 to 80% v of H2, 20-50% v of H20 and some possible gas impurities coming from seal system of the shaft or present in the hydrogen stream 40. When the H2 amount in the reducing gas varies and the compound 10 is charged, the top gas 20 will have an intermediate composition between the two previously described cases.

In an embodiment of the method according to the invention, the top reduction gas 20 after a dust and mist removal step in a cleaning device 5, such as a scrubber and a demister, is sent to a separation unit 6 where it is divided into two streams 22,23. The first stream 22 is a CO2-rich gas which can be captured and used in different chemical processes. In a preferred embodiment, this CO2-rich gas 22 is subjected to a methanation step. The second stream 23 is a H2-rich gas which is sent to a preparation device 7 where it will be mixed with other gas, optionally reformed and heated to produce the reducing gas 11. In a preferred embodiment, the preparation device 7 is a heater.

The method according to the invention allows to obtain a DRI product having enough carbon content without impairing the CO2 footprint of the process.

What is claimed is:

1-9. (canceled)

10. A method for manufacturing direct reduced iron, the method comprising:
reducing oxidized iron in a direct reduction furnace by a reducing gas, the oxidized iron being first mixed with biochar to form a solid compound; and
charging the solid compound into the direct reduction furnace.

11. The method as recited in claim 10 wherein said biochar is produced by the pyrolysis of biomass.

12. The method as recited in claim 10 wherein the solid compound is a briquette or pellet.

13. The method as recited in claim 10 wherein the reducing gas includes more than 50% in volume of hydrogen.

14. The method as recited in claim 10 wherein the reducing gas includes more than 99% in volume of hydrogen.

15. The method as recited in claim 13 wherein the hydrogen of the reducing gas is at least partly produced by electrolysis.

16. The method as recited in claim 15 wherein the electrolysis is powered by renewable energy.

17. The method as recited in claim 10 wherein a top reduction gas is captured at an exit of the direct reduction furnace and subjected to at least one separation step so as to be split between a CO2-rich gas and an H2-rich gas, said H2-rich gas being at least partly used as the reducing gas.

18. The method as recited in claim 17 wherein the CO2-rich gas is subjected to a methanation step.
